(19) United States
(12) Patent Application Publication
(10) Pub. No.: US 2022/0022567 A1
(43) Pub. Date: Jan. 27, 2022

(54) APPARATUS AND METHOD FOR ADJUSTING FIT OF APPAREL

(71) Applicant: Stoko Design, Inc., Vancouver (CA)

(72) Inventors: Nick Rutckyj, Vancouver (CA); Andrew Campbell, Squamish (CA); Kevin Reilly, Vancouver (CA)

(21) Appl. No.: 17/385,615

(22) Filed: Jul. 26, 2021

Related U.S. Application Data
(63) Continuation-in-part of application No. 17/222,903, filed on Apr. 5, 2021.
Application No. 17/222,903 is a continuation-in-part of application No. 16/677,268, filed on Nov. 7, 2019.
Application No. 16/677,268 is a continuation-in-part of application No. 16/216,668, filed on Dec. 11, 2018.
(60) Provisional application No. 63/005,974, filed on Apr. 6, 2020.
(60) Provisional application No. 62/758,549, filed on Nov. 10, 2018.
(60) Provisional application No. 62/640,513, filed on Mar. 8, 2018.
(60) Provisional application No. 62/599,675, filed on Dec. 15, 2017.

(51) Int. Cl. A41D 13/00

(52) U.S. Cl. CPC A41D 13/0007; A41D 2400/38

(57) ABSTRACT

The present invention involves a fit-adjustable item of apparel for a wearer comprises a plurality of tension members each disposed along a corresponding three-dimensional spatial path through the item of apparel, each tension member substantially inextensible along a longitudinal direction of the tension member; a tensioner disposed on the item of apparel and arranged for adjusting tension in the plurality of tension members; and a tension evener disposed on the item of apparel tension-wise in communication with the tensioner and the plurality of tension members, the tension evener arranged for evening tension among the plurality of inextensible tension members, wherein each of the plurality of tension members is arranged in or on the item of apparel to adjust at least one dimension of the item to the wearer by adjusting the tension in the plurality of tension members via the tension evener by operating the tensioner. The tension-evening arrangement is also employed to maintain a device at a targeted location on the body of a wearer.

### CROSS-REFERENCE TO RELATED APPLICATIONS

The present application claims priority under 35 U.S.C. § 120 as a continuation-in-part of U.S. patent application Ser. No. 17/222,903 filed Apr. 5, 2021, which claims priority under 35 U.S.C. § 119(e) from U.S. Provisional Patent Application Ser. No. 63/005,974, filed Apr. 6, 2020; and is further related to and claims priority under 35 U.S.C. § 120 as a continuation-in-part of U.S. patent application Ser. Nos. 16/216,668 and 16/677,268, Ser. No. 16/216,668 filed Dec. 11, 2018 which claims priority under 35 U.S.C. § 119(e) from U.S. Provisional Patent Applications Ser. Nos. 62/599,675 and 62/640,513; filed Dec. 15, 2017, and Mar. 8, 2018, respectively, and Ser. No. 16/677,268 filed Nov. 7, 2019, which claims priority under 35 U.S.C. § 119(e) from U.S. Provisional Patent Application Ser. No. 62/758,549, filed Nov. 10, 2018; the disclosures of each of which are incorporated in their entirety herein.

### BACKGROUND OF THE INVENTION

### Field of the Invention

This present invention relates to apparatus and associated methods for adjusting the size and fit of apparel and garments to the human body. In one aspect, it relates to an apparatus for the automatic evening of tension within size-adjustable or fit-adjustable portions of garments and apparel.

### Description of the Related Art

Garments are generally designed and fabricated to accommodate a particular size or type of individual. For example, an infant's garment may be designed to fit an infant at generally 30 months of age. Thus, the infant's garment is designed to fit an infant having the typical physical measurements of a majority of infants at 30 months of age. However, physical measurements of individuals change over time, especially those of children. In fact, an infant may experience a substantial increase in length in a mere matter of weeks. The infant can therefore rapidly outgrow recently purchased articles of clothing, making those articles of clothing obsolete.

Maternity produces similar challenges. Expectant mothers experience changes in physical proportions both before and after childbirth. Several different sizes of garments are required to accommodate changes in physical proportions throughout maternity, resulting in costly additions to an individual's wardrobe.

Over and above these well-known examples of size-change challenges, there are other areas of human endeavor where the exact fit of garments or other apparel is important. One of those fields is that of orthopaedic wear, where the usual balancing role of supply and demand does not work optimally for the user, and he or she is faced with using orthopaedic wear that has a less than optimal fit.

Another field in which the size and fit of apparel has major effects, is that of backpacks and satchels and the like. A bad fit of these items of apparel can lead to fatigue in and physiological damage to the user.

Attempts to address the general matter of size adjustability and fit adjustability have led, for example, to garments with adjustable girth at the waist. One solution has been to have a waist section that can be expanded or contracted for fitting different waist sizes. The waist section can progressively expand for accommodating waist size changes. Some proposed garments have an elastic band that extends through a tubular casing at the waist section of the garment. A free end of the elastic band is selectively attachable to respective fasteners that are spaced apart along the waist section of the garment. Other art discloses a V-shaped insert panel to cover an opening that is present upon unbuttoning a button-fly of a denim garment. Yet other implementations have gathered waist sections that are held in a gathered position by miniature hook and loop fasteners, while others have an adjustable waist with an opening intersecting a waist section of the garment, a panel bridging across the opening, and a closure mechanism, the opening being narrowed and held by operation of the closure mechanism to narrow the opening, and the opening being widened and held by operation of the closure mechanism, thereby to adjust the girth of the waist section.

Adjustable backpacks and the like are often adjusted by means of a range of straps and buckles or hook and loop fasteners that strive to adjust the fit of the apparel. These often fail for lack of balancing the forces on the body of the user.

Orthopedic braces are used to stabilize joints between the limbs of the human anatomy in cases where the joints or the limbs articulating about them have sustained damage. Braces have been employed to stabilize knees, ankles, elbows and wrists in this way. The brace is applied to reduce strain on the injured limb or joint while permitting the limb or joint to still perform its function, thereby minimizing the risk of further damage. Prior art orthopaedic braces vary greatly in technology, function, and efficacy and generally do not allow adequately for proper user size-fitting or for keeping the forces on the anatomy balanced.

There remains a need for a general means of adequately balancing the forces employed in size-adjustable and fit adjustable garments and apparel. Even simple lacing arrangements exhibit problems with force distribution in such garments and apparel.

### SUMMARY OF THE INVENTION

An item of apparel for a wearer is presented, the item comprising: a plurality of tension members each disposed along a corresponding three-dimensional spatial path through the item of apparel, each tension member substantially inextensible along a longitudinal direction of the tension member; a tensioner disposed on the item of apparel and arranged for adjusting tension in the plurality of tension members; and a tension evener disposed on the item of apparel tension-wise in communication with the tensioner and the plurality of tension members, the tension evener arranged for evening tension among the plurality of inextensible tension members, wherein each of the plurality of tension members is arranged in or on the item of apparel to adjust at least one dimension of the item to the wearer by adjusting the tension in the plurality of tension members via the tension evener by operating the tensioner.

The tension evener may comprise a whippletree. The whippletree may be a multi-tier whippletree. The whippletree may comprise threadable members disposed for engaging with the substantially inextensible strands of the tension member. The threadable members may include one or more of beads, blocks, rings, eyelets and whipple bars disposed for engaging with the plurality of substantially inextensible tension members, wherein the one or more of beads, blocks, rings, eyelets and whipple bars may be made of one of wood, metal, and a polymer, wherein polymer threadable members are one of injection-molded and three-dimensionally printed.

The whippletree may comprise at least one loop of one of substantially inextensible fabric and substantially inextensible cable disposed for engaging with the plurality of tension members. Each of the plurality of tension members may be disposed at least in part in a corresponding conduit, the conduit disposed on or within the item of apparel. The item of apparel may be made of a material having a coefficient of friction and wherein each conduit has a lower coefficient of friction with respect to the tension members than the coefficient of friction of the material of the item of apparel with respect to the tension members. Each conduit may comprise at least in part a tube of material different from the matrix material of the item of apparel.

The whippletree may comprise threadable members disposed for engaging with the substantially inextensible strands of the tension member. The threadable members may include one or more of beads, blocks, rings, eyelets and whipple bars disposed for engaging with the plurality of substantially inextensible tension members, and wherein the one or more of beads, blocks, rings, eyelets and whipple bars may be made of one of wood, metal, and a polymer, wherein polymer threadable members are one of injection-molded and three-dimensionally printed. The whippletree may comprise at least one loop of one of substantially inextensible fabric and substantially inextensible cable disposed for engaging with the plurality of tension members.

The item of apparel may be a fit-adjustable garment. The item of apparel may comprise a fit-adjustable harness. The item of apparel may be a fit-adjustable orthopaedic brace.

A garment is further presented comprising a location-specific apparatus physically attached to the garment in a desired location corresponding to a target location on a body of a wearer of the garment; a tension evener comprising tension members; and a tensioner arranged to adjust a tension in the tension evener, the tension evener disposed and arranged to even tension in the tension members and maintain the location-specific apparatus at the target location. The location-specific apparatus may include orthopaedic braces, inflatable bladders, heat packs, cold packs, heaters, coolers, biometric devices, harnesses and safety devices. The biometric devices may include sensors.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1 is a drawing showing a garment comprising a facility for adjusting the size of the garment.

FIG. 2A to 2C are drawings of embodiments of conduits for use in guiding inextensible members in the garments and apparel described herein.

FIG. 3A to 3F are drawings of embodiments of tension eveners based on two-tier whippletrees for use in evening the tension in inextensible tension members in the garments and apparel described herein.

FIG. 4A to 4C are drawings of further embodiments of tension eveners based on three-tier whippletrees for use in evening the tension in inextensible members in the garments and apparel described herein.

FIG. 5 is a drawing of an adjustable-fit garment comprising a tension-evening arrangement.

FIG. 6 is a drawing of an adjustable-fit backpack comprising a tension-evening arrangement.

FIG. 7 is a drawing of an adjustable-fit backpack comprising a tension-evening arrangement employing a multi-tier whippletree.

FIG. 8 is a drawing of a garment having a sensing or other apparatus located over a targeted area of the body of a wearer and maintained in that position by a whippletree tension evener adjustable via a tensioner.

Corresponding reference characters indicate corresponding parts throughout the several views. Although the drawings represent embodiments of the present invention, the drawings are not necessarily to scale and certain features may be exaggerated in order to better illustrate and explain the present invention. The flow charts and screen shots are also representative in nature, and actual embodiments of the invention may include further features or steps not shown in the drawings. The exemplification set out herein illustrates an embodiment of the invention, in one form, and such exemplifications are not to be construed as limiting the scope of the invention in any manner.

### DESCRIPTION OF EMBODIMENTS OF THE PRESENT INVENTION

The embodiments disclosed below are not intended to be exhaustive or limit the invention to the precise form disclosed in the following detailed description. Rather, the embodiments are chosen and described so that others skilled in the art may utilize their teachings.

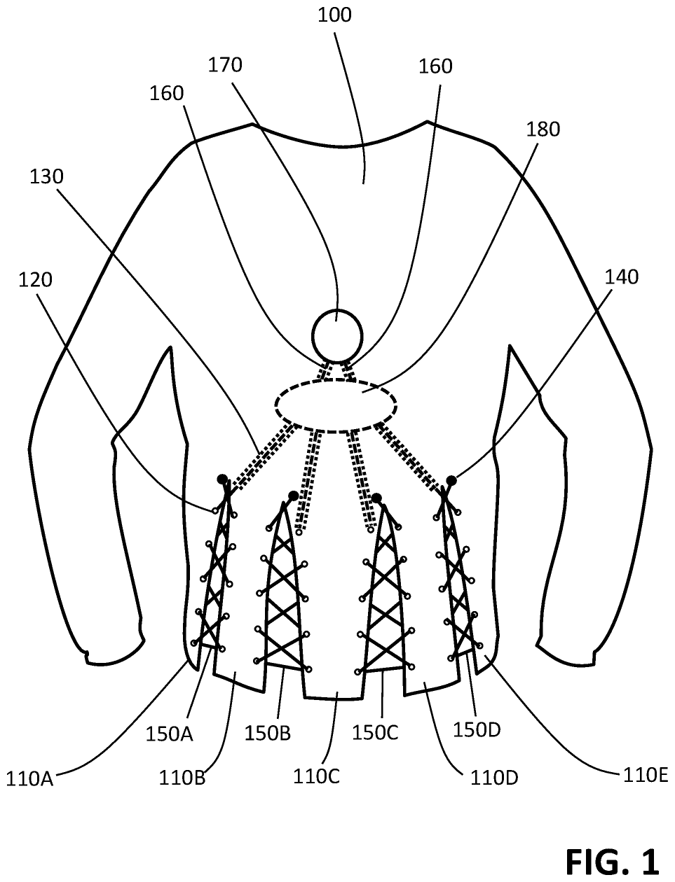

The present invention relates to an item of apparel comprising a mechanism for adjusting the size of the item of apparel. The phrase “item of apparel” is used in this application to describe any item of clothing, garment, or, without limitation, any general item of apparel, such as backpack, harness, and the like, to be worn by or strapped to the body of a wearer. In one embodiment, shown in FIG. 1, coat 100 of adjustable girth comprises a plurality of partially separated coat portions 110A, 110B, 110C, 110D, and 110E, arranged to be pulled together by cords 150A, 150B, 150C and 150D laced through eyelets 120 in coat portions 110A-E. For the sake of clarity and reduction of clutter, only one eyelet is labeled in FIG. 1. Cords 150A-150D are substantially inextensible tension members made of materials described below. Cords 150A-150D may be routed at least in part within conduits 130 disposed within or on garment 100. Conduits 130, shown in broken lines, will be described in more detail below, but may have a curved shape to aid in the distribution of tension in the garment.

Tension in cords 150A-150D may be adjusted by operating tensioner 170. To this end, tensioner 170 communicates tension-wise with tension evener 180 via substantially inextensible tension member 160. Tension member 160 may also be routed via conduits 130 disposed within or on garment 100. For the sake of clarity and reduction of clutter, only one conduit 130 is labeled in FIG. 1. In the particular embodiment shown in FIG. 1, the four ends of cords 150A-150D are individually fixed to four tension member fixing points 140 on garment 100. For the sake of clarity and reduction of clutter, only one fixing points 140 is labeled in FIG. 1.

The tension applied via tensioner 170 to inextensible tension member 160 is transferred within tension evener 180 to inextensible tension members 150A-150D in a manner that will be clear from subsequent drawings and text that follows. In FIG. 1, tension evener 180 is depicted in broken outline as a schematic element without internal structure. It is shown in broken outline because it may, in some embodiments, be incorporated within garment 100, rather than on its exterior. Tension evener 180 is shown without interior structure, because subsequent drawings will offer a variety of implementations of tension evener 180. We treat tension evener 180 in FIG. 1 as an engineering “black box”, still to be described in detail. At this point it is sufficient to note that, in the embodiment shown in FIG. 1, tension members 150A-150D separately extend into tension evener 180 and that tension member 160 also extends through tension evener 180. Tension member 160 may be at both of its ends engaged with tensioner 170, or one of its ends may be fixed to garment 100. In the embodiment shown in FIG. 1, tension member 160 is shown as engaged with tensioner 170 at both of its ends.

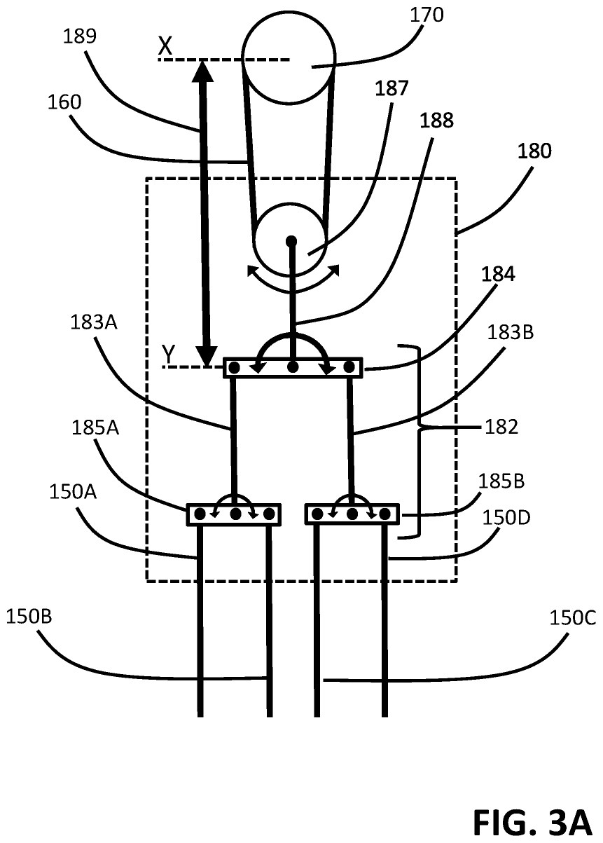

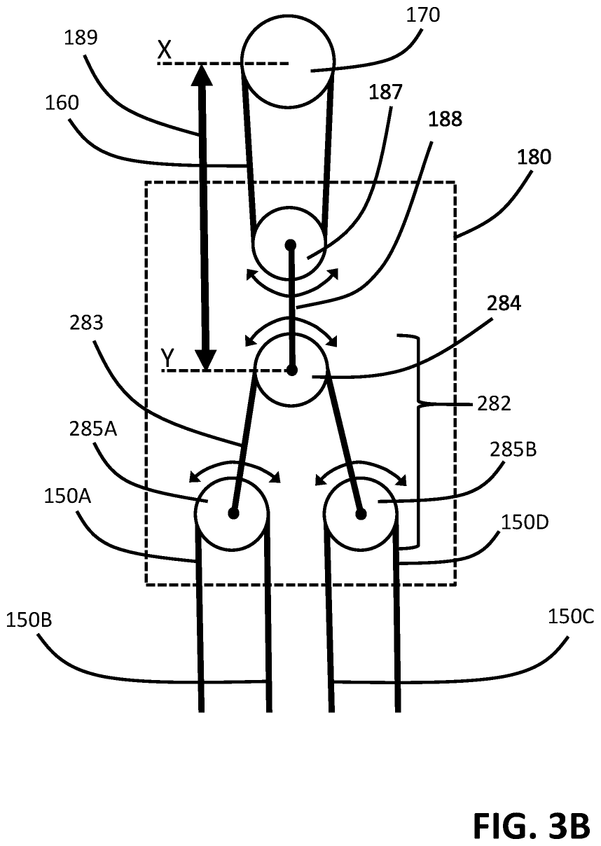

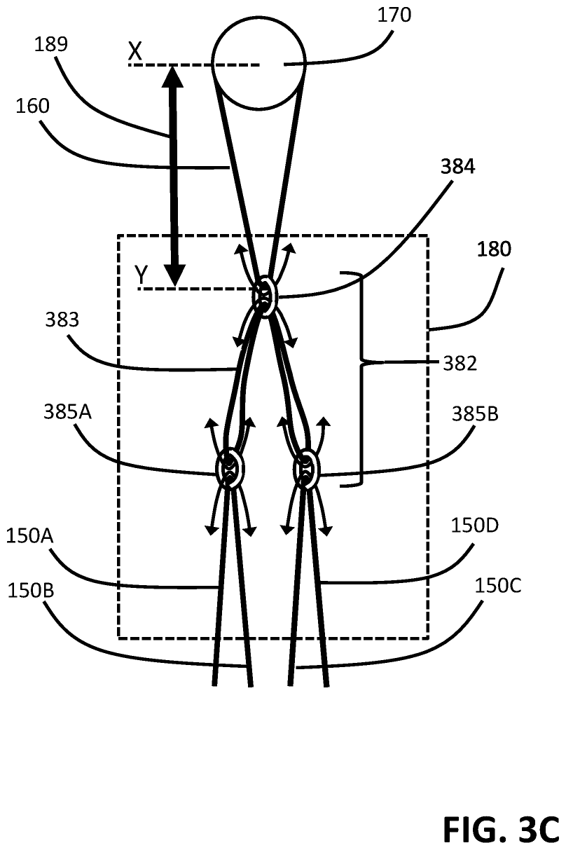

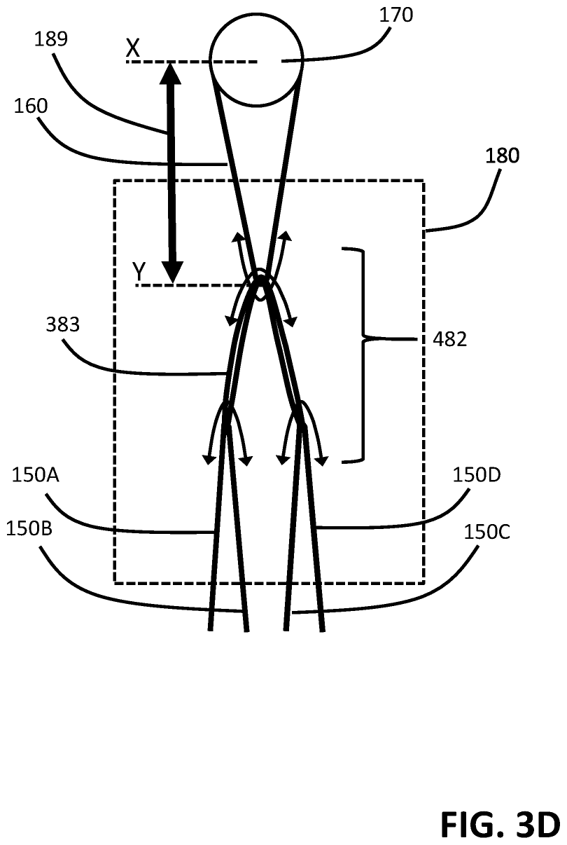

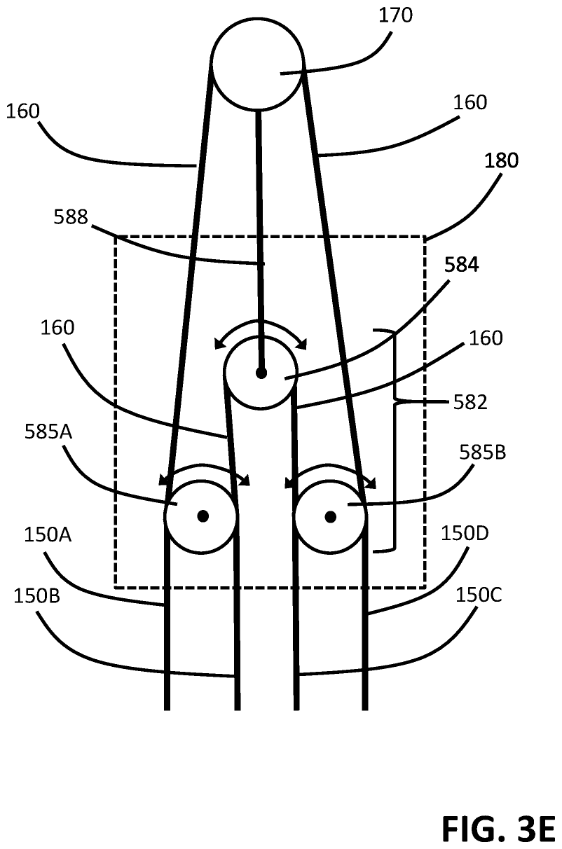

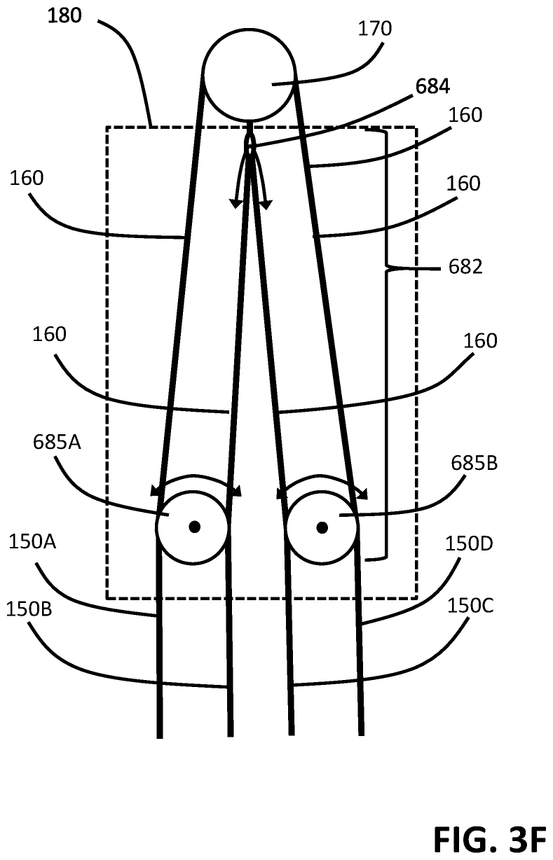

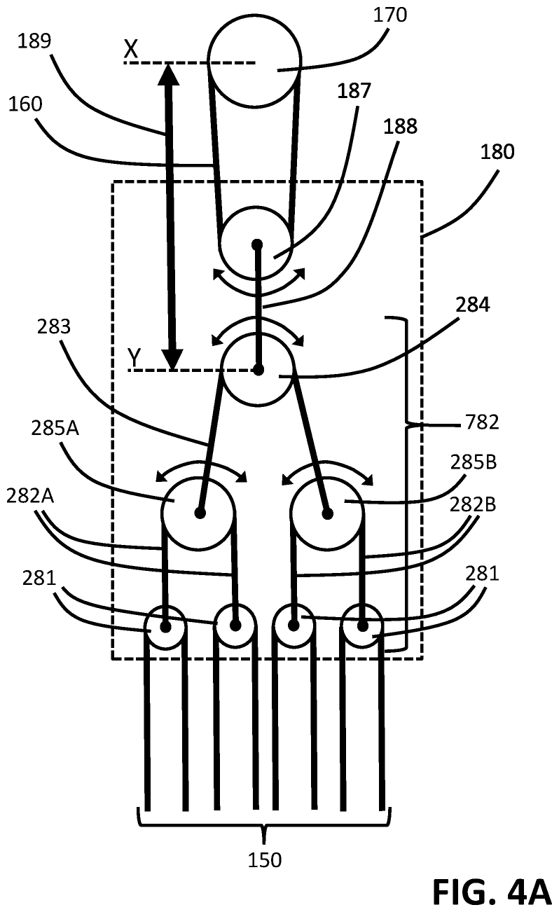

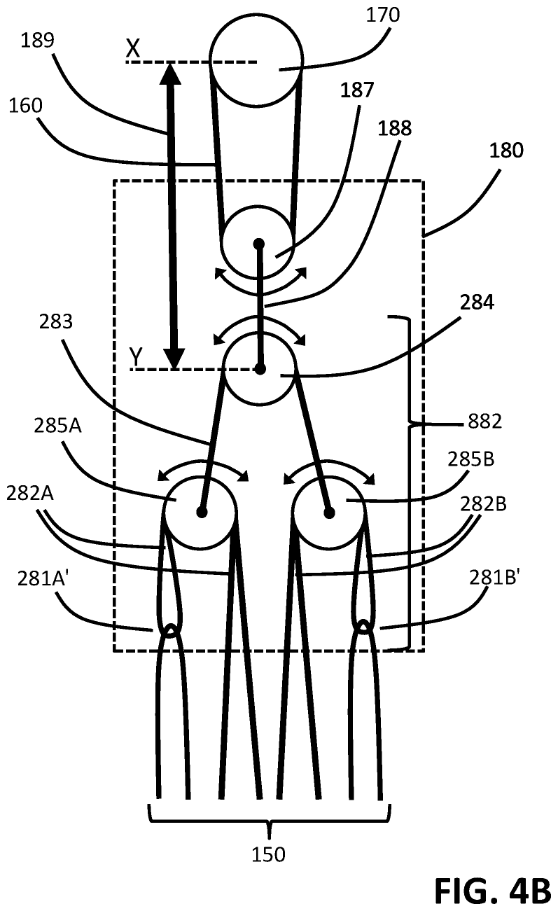

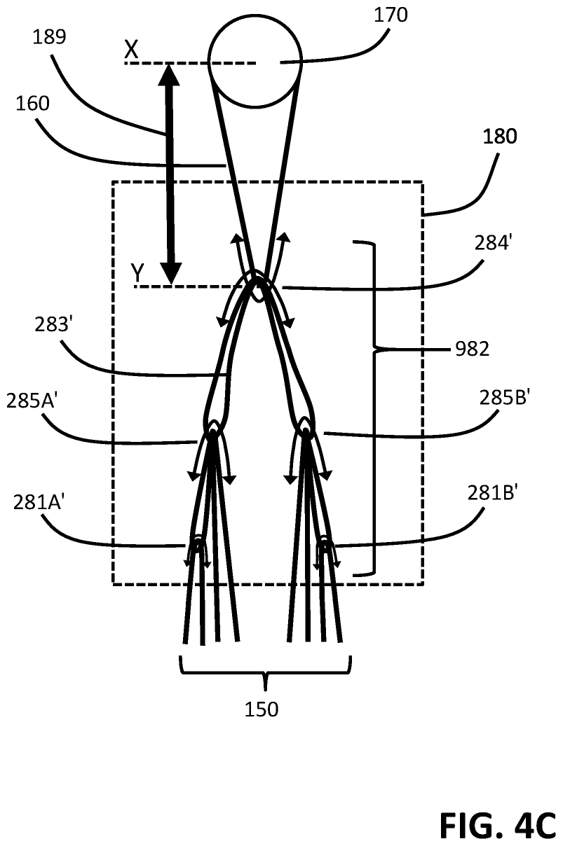

If cords 150A-150D were simply put under increasing tension without any attention to how garment 100 distributes the forces induced by that tension, cords 150A-150D would cause portions 110A-E of coat 100 to crumple and bundle, and the fit of garment 100 would be destroyed. The presence of tension evener 180 ensures that any tension in cords 150A-150D, is evened among cords 150A-150D by the action of tension evener 180, as described further below in FIGS. 3A to 3F and 4A to 4C which show embodiments of tension eveners suitable for this purpose.

Tension members 150A-150D and 160 may be formed of a material that is substantially less extensible than the fabric matrix of garment 100. Materials suited for use in tension members 150A-150D and 160 may be longitudinally inextensible but flexible. Suitable materials for tension members 150A-150D and 160 include, but are not limited to, stainless steel; Nylon; Kevlar®; Teflon®; ultra-high molecular weight polyethylene-based fiber such as Dyneema®; and various fibers with a diamond or basket weave including cotton, polyester and polypropylene, for example without limitation Technora®. (Kevlar is a registered trademark of E. I. DU PONT DE NEMOURS AND COMPANY CORPORATION DELAWARE Chestnut Run Plaza, 974 Centre Road Wilmington, Del. 19805; Teflon is a registered trademark of THE CHEMOURS COMPANY FC, LLC LIMITED LIABILITY COMPANY DELAWARE 1209 ORANGE STREET Wilmington, Del. 19801; Dyneema is a registered trademark of DSM IP Assets B.V. LIMITED LIABILITY COMPANY NETHERLANDS Het Overloon 1 HEERLEN NETHERLANDS NL6411 TE; and Technora is a registered trademark of TEIJIN KABUSHIKI KAISHA (TEIJIN LIMITED) CORPORATION JAPAN 6-7, MINAMIHOMMACHI 1-CHOME CHUO-KU, OSAKA JAPAN).

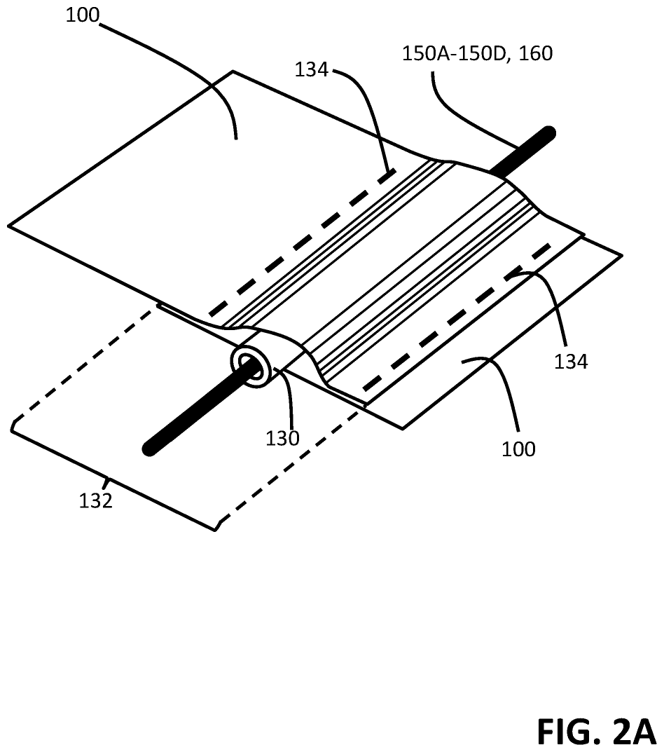

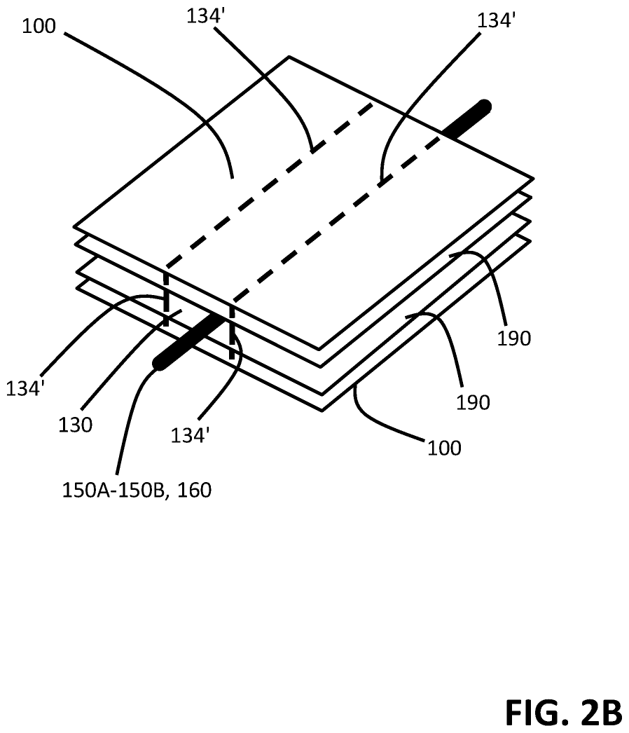

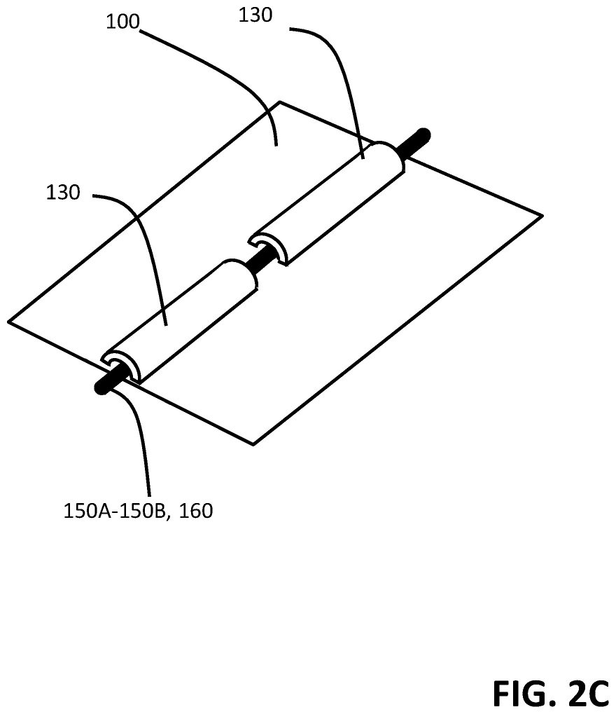

Tension members 150A-150D and 160 may extend at least in part along individual conduits 130 in garment 100, as shown in FIG. 2A, 2B, and 6C. Conduits 130 may be formed of the same material as the matrix fabric of the garment and be lined internally with a conduit material of lesser friction coefficient with respect to the tension member material so as to allow the tension members 150A-150D and 160 to move as freely as possible. The conduit material may be in the form of a tube or a collated tube. There is no particular structural limitation on the cross-section of the tube. A collated tube is useful in that it allows a very low friction tube material to be selected even if it is inextensible, because the collation segments may move closer to one another without unduly crumpling garment 100 when tension members 150A-150D and 160 are put under tension.

FIG. 2A, FIG. 2B, and FIG. 2C show different implementations of conduits for use with tension members. Using the elements of FIG. 1 as example, FIG. 2A shows seam 132 in garment 100 material matrix created by stitches 134, with conduit 130 extending along seam 132 in the form of a tube in this embodiment. Tension members 150A-150D, 160 extend longitudinally through conduit 130. The material of conduit 130 is selected to have a lower coefficient of friction than the general matrix fabric of garment 100 with tension members 150A-150D, 160. In FIG. 2A, conduit 130 is shown as circular in cross-section, but in other embodiments it may have any suitable cross-section that allows 150A-150D, 160 to move substantially freely, while simultaneously making conduit 130 compatible with the ergonomic requirements to which the garment is subject. One suitable cross-section is semi-circular, or a smaller segment of a circle with enough curvature to accommodate tension members 150A-150D, 160, while being flat on one side so as to be easily integrated in garments 100. It has already been explained that conduit 130 may be collated, which allows very low friction, but inextensible tubing to be employed. Example materials for the tubing include, but are not limited to Teflon® and silica. In a related embodiment, tension members 150A-150D, 160 are simply sandwiched between two strips of low fiction material inside a stitched seam. In a further embodiment, seams may be created by joining adjacent material matrices by an adhesive, heat sealing, or other conjoining mechanism, and a conduit formed between suitable spaced-apart seams.

FIG. 2B shows another embodiment of a way to incorporate tension members 150A-150D, 160 within the fabric matrix material of garment 100. The drawing is an edge-on view of the weave of garment 100. It shows two rows 134′ of stitching or weave either side of tension member 150A-150D, 160 within the matrix material of garment 100. Tension member 150A-150D, 160 is sandwiched between two longitudinal strips 190 of low friction material held by stitching 134′. In this drawing, the material of garment 100 is shown as being two sheets. This should be considered as presented schematically for the sake of clarity, as the material is in practice woven. The fiber of the stitching may be a low friction material so that tension member 150A-150D, 160 slides on all sides against low friction material. In this embodiment, conduits 130 are formed by low friction material strips 190 and low friction material stitching 134′. This embodiment may be implemented over the any portion of tension member 150A-150D, 160 that requires guidance for reasons of managing tension.

FIG. 2C shows an embodiment in which conduit 130 is attached to the surface of garment 100 in the form of collated semi-cylindrical tubes and tension member 150A-150D, 160 is routed through conduit 130. In some embodiments, the bases of the tubes may be closed or may be lined with a low friction material. In FIG. 2A, FIG. 2B, and FIG. 2C tension member 150A-150D, 160 is shown as circular in profile or cross-section. In a general embodiment, there is no limitation on the profile or cross-section of tension member 150A-150D, 160.

Garment 100 may comprise segments of “substantially inextensible fabric”. This may include, for example, portions of garment 100 in which tension members 150A-150D and 160 are guided via conduits 130. In the present specification, the term “substantially inextensible fabric” is used to describe a fabric extending in two dimensions that is substantially inextensible in at least a first direction. The fabric may or may not have a restriction on extensibility in a direction perpendicular to the first direction. Non-limiting examples of such materials include but are not limited to so-called 2-way-stretch fabrics with blends of materials including Spandex, Nylon, Dyneema®, Kevlar®, polyester, Ingeo®, olefin fibre, Lyocell, and/or cotton which are woven, knitted, or braided in such a fashion to allow stretch in required dimensions. (Ingeo is a registered trademark of NATUREWORKS LLC LIMITED LIABILITY COMPANY DELAWARE 15305 MINNETONKA BLVD Minnetonka Minn. 55345). The “2-way” stretching refers here to stretching in two opposing directions in a first dimension whilst remaining substantially inextensible in any direction perpendicular to the first direction. Other “substantially inextensible fabrics”, including for example without limitation materials comprising Dyneema® fibers in a two-dimensional mesh embedded in a second fabric or in a sandwich structure, may have substantially no extensibility in any direction. The inextensibility is deemed “substantial” in comparison with the inextensibility of the matrix material of the fabric of garment 100, the garment fabric being stretchable or extensible in comparison with the “substantially inextensible fabric”.

FIG. 3A shows one schematic embodiment of tension evener 180 of FIG. 1 in more detail. Tension evener 180 may comprise whippletree 182, a device known in basic form from the era of the horse-drawn cart. Inextensible cable 188 joins pulley 187 to a fulcrum or pivot point of primary pivoting bar 184 of whippletree 182. The term “whipple bar” is used herein to describe a pivoting bar of a whippletree. The term “swingle” has also been used in some quarters to describe a pivoting bar of the type employed in a whippletree. In other embodiments, inextensible cable 160 (see FIG. 1) may be engaged directly with whippletree 182, this being an embodiment in which pulley 187 is obviated. Whippletree 182 is described in more detail below. Various arrangements of pulleys and inextensible cables may be employed to move whippletree 182 closer to and further away from tensioner 170.

Whippletree 182, as shown in the particular embodiment of FIG. 3A, is a “doubletree” whippletree, being comprised of two tiers of whipple bars, being bar 184 in a first tier, and bars 185A and 185B in the second tier, in order to accommodate four inextensible tension members 150A-D described in FIG. 1. In FIG. 3A, four inextensible members 150A-D are shown connected to the second tier of whipple bars in FIG. 3A. In a more general embodiment, whippletree 182 may have further tiers of whipple bars and engage more tension members. The term “multi-tier whippletree” is used herein to describe a whippletree having a plurality of tiers of whipple bars.

Whipple bar 184 is free to rotate about its fulcrum or pivot point as shown by the rotary arrow associated with whipple bar 184. If the tension in tension member 183B should be higher than the tension in cable 183A, whipple bar 185B will exert a greater force on whipple bar 184 than whipple bar 185A, and whipple bar 184 will pivot clockwise about its pivot point to a degree of rotation at which the difference in tension is evened out. The same action takes place if two tension members 150A and 150B attached to whipple bar 185A are under different amounts of tension. Whipple bar 185A pivots about its indicated pivot point to a degree of rotation at which the tension in the two tension members attached to whipple bar 185A are equal. A similar behavior also holds for whipple bar 185B in respect of any difference in tension between two tension members 150C and 150D attached to whipple bar 185B.

Tensioner 170 is disposed to move the assemblage of pulley 187 and whipple bars 184, 185A and 185B along the direction indicated by bidirectional arrow 189. This is achieved by retracting or extending tension member 160. To this end, one of two ends of tension member 160 may be fixedly attached to tensioner 170 while the other end is retracted or extended by operating tensioner 170. In other embodiments, both ends of tension member 160 may be retracted or extended by operating tensioner 170.

Any integer number N>1 of tension members may be connected to whippletree evener 180 to balance tension by this general mechanism. To address three tension members similar to tension members 150A-150D, two of those tension members may be attached to whipple bar 185A, while whipple bar 185B may be omitted and the third tension member attached directly to whipple bar 184. To employ more than four tension members, an additional tier of whipple bars may be disposed between tension members 150A-D of FIG. 3A and whipple bars 185A and 185B of FIG. 3A, resulting in a “tripletree” whippletree. There is no conceptual limit to the number of tension members that may have their tension balanced in this way by a generalized whippletree evener.

In FIG. 3A, lever-and-fulcrum-based whippletree 182 is employed to describe the general mechanism, but in other embodiments of tension evener 182 arrangements of other devices allowing a tension member to move or slide over or through them may be employed, including devices with holes through which the tension members may pass. Suitable devices of this type include without limitation pulleys, threadable beads, rings, eyelets, blocks, loops of inextensible fabric, and loops in other cables or tension members. The term “block” is used herein to specifically describe a single- or double-holed block as used in a “block and tackle”. The devices may have a connecting mechanism that allows them to be engaged with another functionally similar device. The mechanism to connect them to another functionally similar device may be a another substantially inextensible tension member. In some embodiments the connection mechanism may be a tension member of the same material as tension member 150A-150D, 160.

In FIG. 3B we turn to an alternative embodiment of tension evener 180 of FIG. 3A. In this embodiment, whipple bars 184, 185A and 185B of FIG. 3A are respectively replaced with pulleys 284, 285A and 285B rotating freely about the same axes as the whipple bars they replace. Together they form whippletree 282. Elements 170, 160, 187, 188 and bidirectional arrow 189 are as already described in FIG. 3B and function in the same way as already described at the hand of FIG. 3A. In other embodiments, inextensible member 160 may be engaged directly with pulley 284 to slide over a portion of pulley 284, this being embodiments in which pulley 187 is obviated. Various arrangements of pulleys and inextensible tension members may be employed to move pulley 284 closer to and further away from tensioner 170.

In FIG. 3B tension members 150A and 150B of FIG. 1 are joined to be a single tension member looped about pulley 285A. In treating tension evener 180 as an engineering “black box”, the single resulting tension member presents itself outside tension evener 180 as the two tension members 150A and 150B of FIG. 1. Tension members 150C and 150D of FIG. 1 are joined to be a single tension member looped about pulley 285B. In treating tension evener 180 as an engineering “black box”, the single resulting tension member presents itself outside tension evener 180 as two tension members 150C and 150D of FIG. 1. Pulleys 285A and 285B are connected by inextensible tension member 283 engaged with pulley 284. In this respect, single tension member 283 replaces tension members 183A and 183B of FIG. 3A. If the tension in portions 150A and 150B of the tension member engaged with pulley 285A is not even, then that tension member either slides over pulley 285A or rotates pulley 285A until the tension is evened out and equal in tension member portions 150A and 150B. Similarly, if the tension in portions 150C and 150D of the tension member engaged with pulley 285B is not even, then that tension member either slides over pulley 285B or rotates pulley 285B until the tension is evened out and equal in tension member portions 150C and 150D.

When the resolved tension in the joint tension member (150A and 150B) engaged with pulley 285A and the resolved tension in the joint tension member (150C and 150D) engaged with pulley 285B are mutually unbalanced, the two pulleys move with respect to each other as tension member 283 slides over pulley 284 or rotates pulley 284 to even out the tension in tension members 150A-D. This tension evening arrangement functions like whippletree 182 of FIG. 3A. The degrees of rotation of the various pulleys are determined by the tension differences already described and the radii of the pulleys. The difference in the embodiment of FIG. 3B is that whipple bars 184, 185A and 185B of FIG. 3A are replaced here by pulleys 284, 285A and 285B, while cables 183A and 183B are replaced by single cable 283. The particular embodiment in FIG. 3B is also a “doubletree” (two-tier) whippletree 282. As a parallel to the arrangement of FIG. 3A, the embodiment of FIG. 3B may be similarly extended to encompass more tiers of pulleys and larger or odd numbers of tension members. In general, the whippletree embodiment in FIG. 3B is another example of a multi-tier whippletree.

A further embodiment of tension evener 180 is shown in FIG. 3C in which the pulleys of FIG. 3B are replaced by rings through which tension members are looped. The rings may be of any suitable material capable of withstanding the applied tension, including but not limited to metal rings, plastic rings and fabric rings or loops of fabric. More specifically, tension member 283 of FIG. 3B is replaced by looped inextensible tension member 383. As in FIG. 3B, tension members 150A and 150B of FIG. 1 are joined to be a single tension member looped through ring 385A. In treating tension evener 180 as an engineering “black box”, the single resulting tension member presents itself outside tension evener 180 as tension members 150A and 150B of FIG. 1. Tension members 150C and 150D of FIG. 1 are joined to be a single tension member looped through ring 385B. In treating tension evener 180 as an engineering “black box”, the single resulting tension member presents itself outside tension evener 180 as tension members 150C and 150D of FIG. 1.

Inextensible tension member 160 applying tension from tensioner 170 is threaded directly through ring 384 and closed loop tension member 383 is also threaded directly through ring 384. Pulley 187 of FIG. 3B is thereby obviated as is tension member 188 of FIG. 3B. In the embodiment shown in FIG. 3C, tension evener 180 and whippletree 382 comprise no pulleys or whipple bars and the working of the device is based exclusively on tension members sliding within rings of the types described above. The functioning is nevertheless similar to that shown in FIG. 3B. As a parallel to the arrangements of FIG. 3A and FIG. 3B, the embodiment of FIG. 3C may be similarly extended to encompass more tiers of rings and larger or odd numbers of tension members. In general, the ring-based whippletree embodiment in FIG. 3C is another example of a multi-tier whippletree.

A further embodiment of tension evener 180, derived from the arrangement in FIG. 3C, is shown in FIG. 3D in which the rings of FIG. 3C are replaced by loops in substantially inextensible tension members, cables, or laces. In this implementation joined tension members 150A and 150B are threaded directly through closed loop tension member 383, and so are joined tension members 150C and 150D. This arrangement obviates all the rings of the implementation shown in FIG. 3C. Looped inextensible cable 383 is threaded directly through inextensible tension member 160 applying tension from tensioner 170 to tension evener 180.

In this embodiment, tension evener 180 and whippletree 482 comprise no rings, pulleys, or whipple bars and the working of the device is based exclusively on tension members sliding within loops of other tension members. The functioning is nevertheless similar to that shown in FIG. 3C. As a parallel to the arrangements of FIG. 3A, FIG. 3B, and FIG. 3C, the embodiment of FIG. 3D may be similarly extended to encompass more tiers of tension members loops and larger or odd numbers of tension members. In general, the loop-based whippletree embodiment in FIG. 3D is another example of a multi-tier whippletree.

Alternative embodiments of tension evener 180 and its interaction with tensioner 170 include the example shown in FIG. 3E. For the sake of clarity of function, we show this embodiment using pulleys as in FIG. 3B. In this embodiment inextensible tension member 160 is also extended and retracted by tensioner 170 to adjust tension in the system, but is engaged with both pulley 585A and pulley 585B. Between engaging with pulley 585A and pulley 585B, tension member 160 also engages with pulley 584. Tension member 588 serves simply to fixedly attach pulley 584 to tensioner 170. As in FIG. 3B, joined tension members 150A and 150B are also engaged with pulley 585A, and joined tension members 150C and 150D are engaged with pulley 585B. Comparison with FIGS. 3B and 3C shows that this arrangement is equivalent to omitting respectively tension member 283 and tension member 383 and assigning their tension evening function to tension member 160 in whippletree 582. The motion of tension member 160 and tension members 150A-D in this embodiment may be a combination of sliding over and rotation of the pulleys they are engaged with. This arrangement remains a two-tier whippletree and is therefore also an example of a multi-tier whippletree. As a parallel to the arrangements of FIGS. 3A, 3B, 3C, and 3D, the embodiment of FIG. 3E may be similarly extended to encompass more tiers of pulleys and larger or odd numbers of tension members.

In FIG. 3F, a variant of the embodiment in FIG. 3E is shown in which tension member 588 and pulley 584 of FIG. 3E are simply replaced with eyelet 684, a label specifically chosen to indicate that eyelet 684 fulfills a similar role in FIG. 3F as pulley 584 does in FIG. 3E. Eyelet 684 may be made from any material that may withstand the tension applied to it. As a parallel to the arrangements of FIGS. 3A, 3B, 3C, 3D, and 3E, the embodiment of FIG. 3F may be similarly extended to encompass more tiers of pulleys and larger or odd numbers of tension members. For example, a further eyelet 684 may be added to tensioner 170 and tension member 160 may engage with a third pulley disposed between pulleys 685A and 685B, the third pulley engaging with another tension member similar to 150A-D. The arrangement 682 in FIG. 3F, and that of its extension described here, remain two-tier whippletrees and are therefore also examples of multi-tier whippletrees. As shown in FIG. 3D with respect to FIGS. 3A, 3B, and 3C, the pulleys of FIGS. 3E and 3F may be replaced by suitably arranged looped tension members. Some embodiments, for example the embodiment shown in FIG. 3E, may have mechanical advantage in that the force exerted on tension member 160 at tensioner 170 results in a greater force on tension members 150A-D.

Returning now to FIG. 1, alternative embodiments may have a larger number of tension members 150 instead of tension members 150A to 150D. We may take the example of eight tension members as described at the hand of FIG. 4A to 4C below.

FIG. 4A shows how alternative tension evener 180 comprising three-tier whippletree 782 may be employed to even the tension in eight tension members 150. In order to avoid cluttering FIG. 4A-4C, we label all tension members on the opposing side of tension evener 180 from tensioner 170 in FIG. 4A-4C as “150”. Eight tension members 150 arise from four tension members being looped over four pulleys 281. Three-tier whippletree 782 may be realized in any of the forms described with reference to FIGS. 3A to 3F. For the sake of clarity, a pulley-based implementation, building on the system of FIG. 3B, is chosen. In this embodiment, two inextensible cables 282A and 282B are fixed at their four ends to four pulleys 281. The eight tension members 150 are engaged with pulleys 281. The tension evening function of this embodiment proceeds as already explained, except that there is in FIG. 4A an additional tier of pulleys 281 and cables 282A and 282B to undertake the balancing of the tension in tension members 150. In all other respects, the embodiment of FIG. 4A is the same as that of FIG. 3B and the remaining elements bear the same labels as in FIG. 3B.

FIG. 4B shows yet another embodiment of three tier whippletree arrangement 882 based on the whippletree arrangement of FIG. 4B. In the embodiment of FIG. 4B, pulleys 284 are absent. Instead, two of four pulleys 281 are functionally replaced by mutual loopings 281A′ and 281W of pairs of continuous tension members 150, while tension members 150 are routed over pulleys 285A and 285B. This remains a three-tier whippletree, though the third tier, unlike that of FIG. 4A, has only two pivot points. To convert the embodiment of FIG. 4B into a whippletree evener of which the third tier would have four pivot points, the same arrangement maybe employed about each of pulleys 285A and 285B as is used for cable 383 of FIG. 3C. In such an arrangement, none of cables 150 would be engaged with a pulley and each would have a looping arrangement such as 281A′ or 281B′, for a total of four.

FIG. 4C shows yet a further embodiment of a three-tier whippletree arrangement 982 based on the two-tier whippletree arrangement of FIG. 3D and employing the looped tension member 150 configuration of FIG. 4B with loopings 281A′ and 281W replacing pulleys 285A and 285B of FIG. 4B respectively. The interfacing of tensioner 170 with tension evener 180 is the same as in FIG. 3D and we therefore employ the labeling of FIG. 3D for the rest of the structure outside evener 180.

As already explained for the two-tier whippletree with reference to FIGS. 3B to 3F, the three-tier arrangements of FIGS. 4A to 4C may also be implemented in other mechanical variants, including rings, eyelets or blocks of suitable materials to withstand the tension, including fabric, in order to serve the same function. The embodiments of FIGS. 4A, 4B and 4C thereby also rely on a multi-tier whippletree tension evener.

In general, the various tension eveners described herein may comprise a whippletree that may be a multi-tier whippletree. The whippletree may more particularly be a two-tier whippletree or a three-tier whippletree and the whippletree may employ one or more of pulleys and threadable members. The threadable members may include one or more of beads, rings, eyelets, blocks, loops of inextensible fabric, loops in inextensible cables, and whipple bars for engaging with the various inextensible members. The pulleys, threadable beads, rings, eyelets, blocks and whipple bars may be formed of any material capable of withstanding the tensions applied, including without limitation wood, metal, plastic and other suitable polymer materials. The elements may be formed by, for example without limitation, injection molding or three-dimensional printing technology.

We have described above the example embodiment of a piece of apparel in the form of a size-adjustable garment, more particularly, coat 100. We have also described various embodiments of the whipple tree tension evener 180 of FIG. 1 and of the conduits that may be employed to guide the various substantially inextensible members, including tension members 160 and 150A-D.

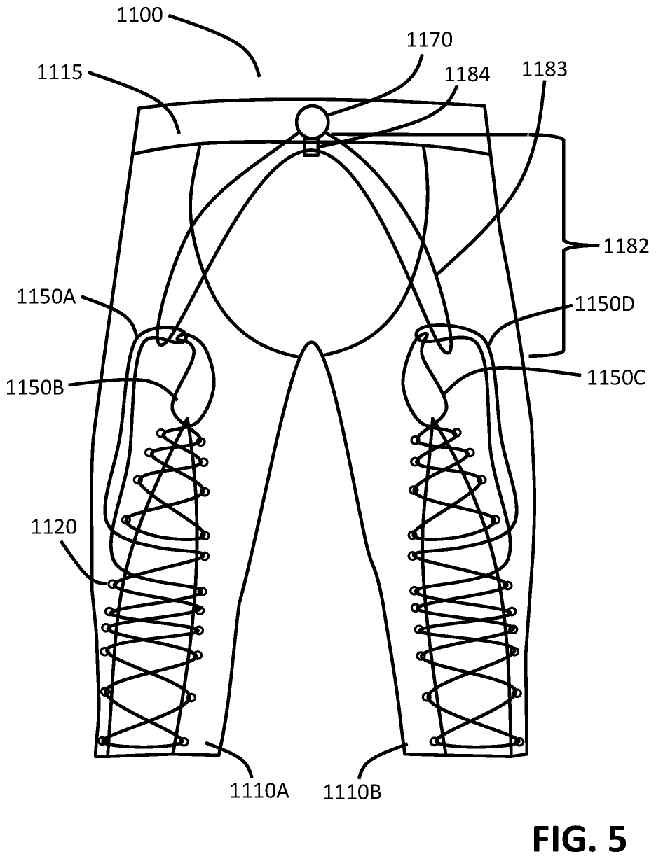

FIG. 5 shows another embodiment of the present invention, being size-adjustable or adjustable-fit garment 1100 configured for wearing on the legs and lower torso of the human body. Garment 1100 is shown with all tension members relaxed for the purposes of clarity. Right and left leg portions 1110A and 1110B of the garment have slits configured to be closed to adjustable degrees. To this end, leg portion 1110A and leg portion 1110B of garment 1100 each has two substantially inextensible tension members in the form of closed loops threaded through eyelets 1120, with the tension members additionally looped through each other. For example, tension member 1150B is looped through tension member 1150A and tension member 1150C is looped through tension member 1150D. In order to avoid cluttering the drawing, only one single eyelet 1120 is labeled in FIG. 5. All tension members in garment 1100 may be disposed at least in part within conduits similar to conduits 130 of FIG. 1. In FIG. 5, any such conduits are not shown in order to avoid cluttering the drawing and obscuring key features.

In the embodiment shown in FIG. 5, both ends of substantially inextensible tension member 1183 are engaged with tensioner 1170. Tensioner 1170 is disposed and configured to adjust a tension in tension member 1183. In a parallel to eyelet 684 of FIG. 3F, threadable member 1184 is affixed on the garment proximate tensioner 1170. With tension member 1183 threaded through threadable member 1184, the arrangement 1182 in FIG. 5 forms a multi-tier whippletree, with at least one of tension member 1150A and tension member 1150B looped through tension member 1183, and at least one of tension member 1150C and tension member 1150D looped through tension member 1183. The lower parts of the whippletree structure 1182 of FIG. 5 has similarities to the lower parts of whipple tree 982 of FIG. 4C. The upper parts of whippletree 1182 of FIG. 5 has similarities to the upper parts of whipple tree 682 of FIG. 3F. Threadable member 1184 may be formed from a substantially inextensible fabric material.

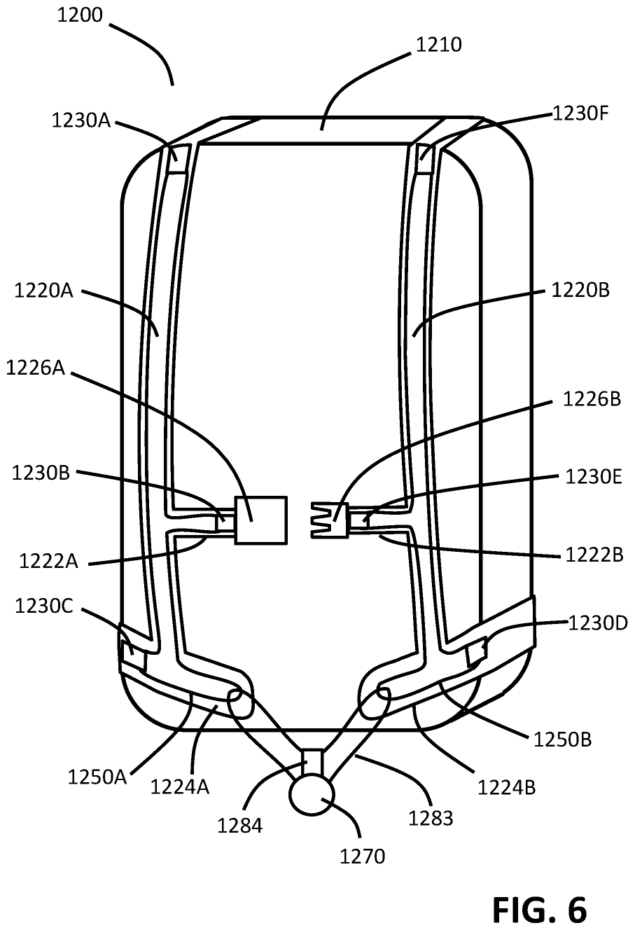

FIG. 6 shows another embodiment of the present invention, being a piece of adjustable-fit apparel in the form of backpack 1200, presented here in very basic form in order to ensure that key features are clear. Backpack 1200 comprises carrier portion 1210 employed as the volume within which items are borne by the user. Backpack 1200 comprises, attached to its right-hand side, a right-hand fitting strap arrangement comprising shoulder strap 1220A, chest strap 1222A and waist strap 1224A. Backpack 1200 comprises, attached to its left-hand side, a left-hand fitting strap arrangement comprising shoulder strap 1220B, chest strap 1222B and waist strap 1224B. Substantially inextensible closed loop tension member 1250A is disposed within shoulder strap 1220A, chest strap 1222A and waist strap 1224A. Closed loop tension member 1250A is looped through threadable members 1230A, 1230B and 1230C. Other configurations of backpacks may be used within the teaching of the present invention, this illustrated embodiment showing one application of the present invention embodying a general backpack design.

Substantially inextensible closed loop tension member 1250B is disposed within shoulder strap 1220B, chest strap 1222B and waist strap 1224B. Closed loop tension member 1250B is looped through threadable members 1230F, 1230E and 1230D. Chest straps 1222A and 1222B may be fastened to each other by any means that provides a substantially inextensible coupling. In the embodiment of FIG. 6, an arrangement is shown employing latch 1226A and latch plate 1226B, similar to that used in automotive seat belts.

Tension members 1250A and 1250B may be disposed at least in part within conduits similar to conduits 130 of FIG. 1, shown in more detail in FIGS. 2A, 2B and 2C. In FIG. 6, no conduits are shown in order to avoid cluttering the drawing and obscuring key features. Six straps 1220A, 1220B, 1222A, 1222B, 1224A and 1224B are all shown as transparent for the sake of clarity. In a general backpack of the design shown in FIG. 6, all straps may be opaque and tension members 1250A and 1250B may in great part be invisible. In other embodiments, the tension members and threadable members may be disposed on the surfaces of the straps and visible from the exterior.

Substantially inextensible tension member 1283 is looped through tension members 1250A and 1250B and through threadable member 1284. In use, the tension members function to produce tension in the straps, thereby to facilitate fitting the backpack to the body of the user. Threadable member 1284 is attached to tensioner 1270. Tension member 1283 is shown as having both its ends engaged with tensioner 1270. Various tensioners are known in the art and, for the purposes of this invention, a winding mechanism is selected. Other tensioners are also usable in this invention. In other embodiments, only one end of tension member 1283 may be engaged with tensioner 1270, while the second end may be merely attached to tensioner 1270 without also being wound by tensioner 1270. When tension member 1283 is tensioned by tensioner 1270, and latch plate 1226B is engaged with latch 1226A, tension member 1283 acts upon tension members 1250A and 1250B, providing tension in tension members 1250A and 1250B. Comparison with FIG. 3A-3F and FIG. 4A to 4C shows that the tension member arrangement of FIG. 6 provides a whippletree tension evener, being merely upside down compared with the devices of FIG. 3A-3F and FIG. 4A to 4C. The whippletree arrangement of FIG. 6 allows the fit of backpack 1200 to the body of the user to be adjusted by adjusting tensioner 1270, the whippletree ensuring that the tension is evenly distributed.

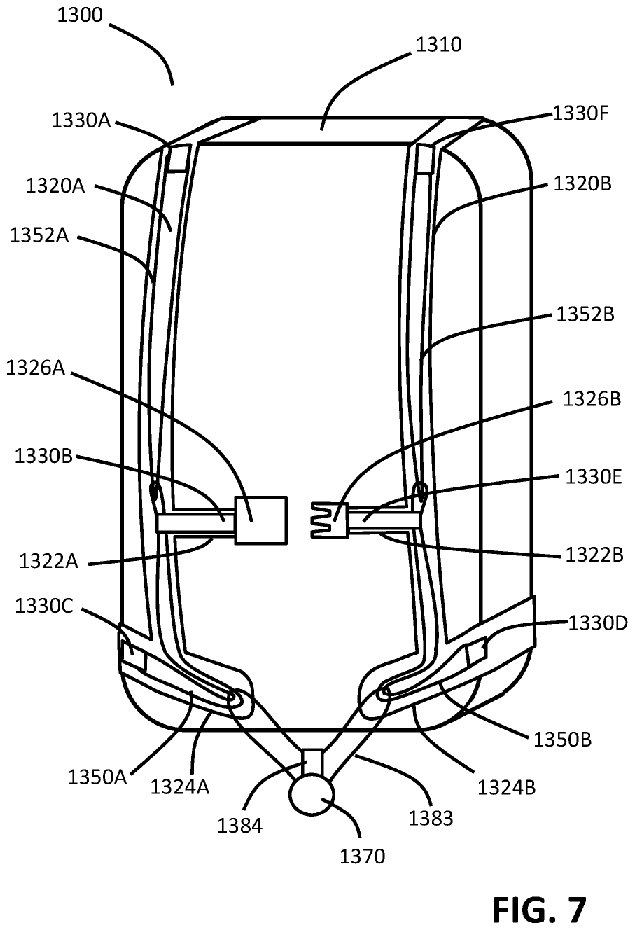

FIG. 7 shows another embodiment of the present invention, being a piece of adjustable-fit apparel in the form of backpack 1300, presented here in very basic form in order to ensure that key features are clear. Backpack 1300 comprises carrier portion 1310 employed as the volume within which items are borne by the user. Backpack 1300 comprises, attached to its right-hand side, a right-hand fitting strap arrangement comprising shoulder strap 1320A, chest strap 1322A and waist strap 1324A. Backpack 1300 comprises, attached to its left-hand side, a left-hand fitting strap arrangement comprising shoulder strap 1320B, chest strap 1322B and waist strap 1324B. Substantially inextensible closed loop tension member 1350A is disposed within shoulder strap 1320A, chest strap 1322A and waist strap 1324A. Closed loop tension member 1350A is looped through threadable members 1330B and 1330C. Substantially inextensible closed loop tension member 1352A is disposed within shoulder strap 1320A and is threaded through threadable member 1330A and through substantially inextensible closed loop tension member 1350A.

In a further embodiment, based on the adjustable-fit apparel of FIG. 7, tension members 1350A and 1350B may be fixedly attached to members 1330C and 1330D respectively, and members 1330C and 1330D are not required to be threadable. Similarly, tension members 1352A and 1352B may be fixedly attached to members 1330A and 1330F respectively, and members 1330A and 1330F are not required to be threadable.

Substantially inextensible closed loop tension member 1350B is disposed within shoulder strap 1320B, chest strap 1322B and waist strap 1324B. Closed loop tension member 1350B is looped through threadable members 1330E and 1330D. Substantially inextensible closed loop tension member 1352B is disposed within shoulder strap 1320B and is threaded through threadable member 1330F and through substantially inextensible closed loop tension member 1350B.

Chest straps 1322A and 1322B may be fastened to each other by any mechanism that provides a substantially inextensible coupling. In the embodiment of FIG. 7, an arrangement is shown employing latch 1326A and latch plate 1326B, similar to that used in automotive seat belts.

Tension members 1350A, 1350B, 1352A and 1352B may be disposed at least in part within conduits similar to conduits 130 of FIG. 1, shown in more detail in FIG. 2A, 2B and 2C. In FIG. 7, no conduits are shown in order to avoid cluttering the drawing and obscuring key features. Six straps 1320A, 1320B, 1322A, 1322B, 1324A and 1324B are all shown as transparent for the sake of clarity. In a backpack of the general design shown in FIG. 7, all the straps may be opaque and tension members 1350A, 1350B, 1352A and 1352B may in great part be invisible. In other embodiments, the tension members and threadable members may be disposed on the surfaces of the straps.

Substantially inextensible tension member 1383 is looped through tension members 1350A and 1350B and through threadable member 1384. In use, the tension members function to produce tension in the straps, thereby to facilitate fitting the backpack to the body of the user. Threadable member 1384 is attached to tensioner 1370. Tension member 1383 is shown as having both its ends engaged with tensioner 1370. Various tensioners are known in the art and, for the purposes of this invention, a winding mechanism is selected. Other tensioners are also usable in this invention. In other embodiments, only one end of tension member 1383 may be engaged with tensioner 1370, while the second end may be merely attached to tensioner 1370 without also being wound by tensioner 1370. When tension member 1383 is tensioned by tensioner 1370, and latch plate 1326B is engaged with latch 1326A, tension member 1383 acts upon tension members 1350A and 1350B, providing tension in tension members 1350A and 1350B. Comparison with FIG. 4C and its associated description shows that the tension member arrangement of FIG. 7 provides a multi-tier whippletree tension evener, being merely upside down compared with the device of FIG. 4C. The whippletree arrangement of FIG. 7 allows the fit of backpack 1300 to the body of the user to be adjusted by adjusting tensioner 3270, the whippletree ensuring that the tension is evenly distributed.

The same general arrangement of tension members, threadable members, tensioner and tension evener as in FIG. 4A to FIG. 7 may be applied to other fit-adjustable and size-adjustable apparel and garments. The backpacks of FIG. 6 and FIG. 7 are merely particular embodiments employing harnesses in the form of the straps in the figures. These arrangements may be extended to harnesses in general, as well as to safety equipment in general. In all cases a plurality of substantially inextensible tension members extends through portions of the equipment with the tension in the tension members balanced by a tension evener that is tension-wise in communication with a tensioner.

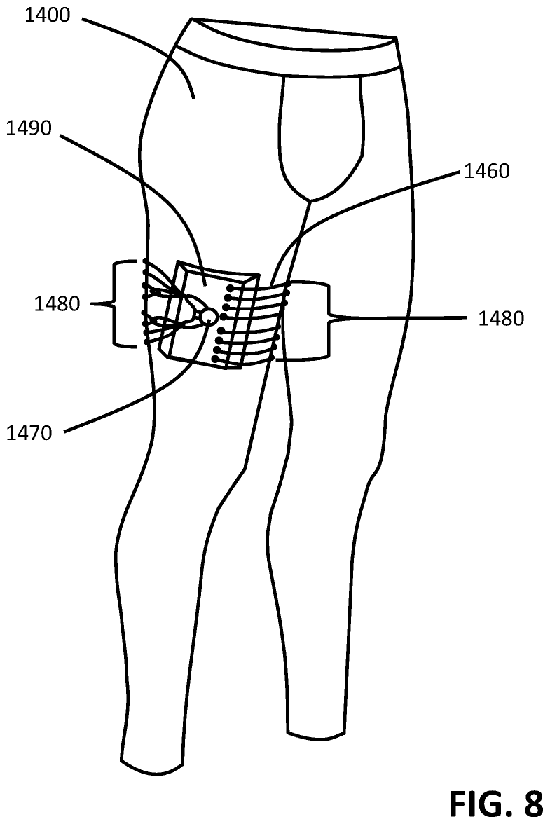

In a further embodiment, shown schematically in FIG. 8, garment 1400 comprises location-specific device or apparatus 1490 physically attached to garment 1400 in a desired location corresponding to a target location on a body of a wearer of garment 1400; a tension evener 1480 engaged with location-specific device 1490 and comprising substantially inextensible tension members 1460; and tensioner 1470 arranged to adjust a tension in tension evener 1480, the tension evener disposed and arranged to even tension in tension members 1460 and maintain location-specific apparatus 1490 at the target location. This arrangement allows tension evener 1480 to maintain the placement of location-specific device 1490 on the body of the wearer despite any flexing of the body of the wearer. It also allows for the adjustment of any force with which the location-specific device 1490 is held against the body of the wearer. Location-specific device 1490 may be positioned between the fabric of garment 1400 and the body of the wearer. In other embodiments location-specific device 1490 may be positioned on the exterior of garment 1400. In this embodiment, garment 1400 is fit-adjustable to the extent that the arrangement allows the fitting of location-specific device 1490 to the body of the wearer by adjusting elements of garment 1400. Tension evener 1480 may be a whippletree tension evener and may be a multitier whippletree. Tension evener 1480 may be, for example, of the type described in FIGS. 1, 3A-3F, or 4A-4C. In FIG. 8, for example, a whippletree tension evener 1480 is shown of the same multitier type as in FIG. 4C and FIG. 5. Other whippletree arrangements may also be employed in the garment 1400 of FIG. 8.

The forms of location-specific device 1490 contemplated as part of this embodiment includes, without limitation, orthopaedic braces, inflatable bladders, heat packs, cold packs, heaters, coolers, biometric devices, harnesses and safety devices. The biometric devices may include sensors. Prior art orthopedic braces tend to migrate during normal wear, leaving users in a compromised state. Furthermore, prior art pads used in protective equipment tend to shift and move around during activity. In addition to this, prior art pads may migrate away from the area they are supposed to protect when contact is made with another player during sport. The whipple tree tensioning system of the present invention may retain the location of the orthopaedic brace through a range of motion while reducing the number and/or extent of migration(s). This ensures that support is in the right place when the user goes into a compromising position, whether worn on the thigh, knee, elbow, ankle or other part of the body. In FIG. 8, location-specific device 1490 in question is shown as located on the right thigh of the user. More than one location-specific device 1490 may be incorporated into garment 1400 and the various pieces of apparatus may be of differing types.

In a further embodiment, based on the braces described in co-pending U.S. patent application Ser. No. 17/222,903 and Patent Cooperation Treaty Application PCT/CA2021/050454, both incorporated herein in full, a brace garment as described in those two applications is fit-adjustable and/or size-adjustable by the same mechanisms as described above in the present application to fit the wearer whilst serving as an orthopaedic brace.

Prior art air bladders inflated against the body of the user tend to shift and move away from the body due to the outwards force caused by the pressure. One application of prior art air bladders next to the body is in athletic recovery apparel. Such air bladders are inflated against the body to provide a compressive force and move fluid from the limbs after an intense workout leading to increases circulation, reduced pain and soreness. An air bladder held next to the body using a whipple tree tensioning system, as in the present invention, allows for adjustability, reduced migration of the air bladders, and ensures that an even force is transferred to the body across the bladder.

It is very difficult to ensure that ice packs and heat packs prior art solutions stay in intended locations on the body. The whipple tree tensioning system of the present invention holds ice/hot packs in place on the body to keep the targeted heat or ice in the correct area on the body of the user. In addition to this, the arrangement of the present invention allows hot or cold apparatus to stay in place on the body while users to move about. Prior art sensors and biometric devices similarly tend to move about on the body of the user, resulting in skewed measurements. The use of the apparatus-locating arrangement of the present invention ensures more consistent locating of the apparatus and thereby more consistent and reliable data.

Safety equipment and harnesses are also prone to reduction in their effectiveness due to the devices badly fitting the user or moving around during use. This is often due at least in part to them being difficult to adjust and the fact that the tend to sit proud of the curved surfaces of the body of the wearer. The use of the tension evener arrangement of the present invention helps to locate, secure, anchor and mitigate migration of safety equipment and harnesses on the body.

While this invention has been described as having exemplary designs, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. An item of apparel for a wearer, the item comprising:
one or more tension members each disposed along a corresponding three-dimensional spatial path through the item of apparel, each tension member substantially inextensible along a longitudinal direction of the tension member;
a tensioner disposed on the item of apparel and arranged for adjusting tension in the plurality of tension members; and
a tension evener disposed on the item of apparel tension-wise in communication with the tensioner and the plurality of tension members, the tension evener arranged for evening tension among the plurality of inextensible tension members, wherein each of the plurality of tension members is arranged in or on the item of apparel to adjust at least one dimension of the item to the wearer by adjusting the tension in the plurality of tension members via the tension evener by operating the tensioner.

2. The item of apparel of claim 1, wherein the tension evener comprises a whippletree.

3. The item of apparel of claim 2, wherein the whippletree is a multi-tier whippletree.

4. The item of apparel of claim 2, wherein the whippletree comprises threadable members disposed for engaging with the substantially inextensible strands of the tension member.

5. The item of apparel of claim 4, wherein the threadable members include one or more of beads, blocks, rings, eyelets and whipple bars disposed for engaging with the plurality of substantially inextensible tension members, and wherein the one or more of beads, blocks, rings, eyelets and whipple bars is made of one of wood, metal, and a polymer, wherein polymer threadable members are one of injection-molded and three-dimensionally printed.

6. The item of apparel of claim 2, wherein the whippletree comprises at least one loop of one of substantially inextensible fabric and substantially inextensible cable disposed for engaging with the plurality of tension members.

7. The item of apparel of claim 1, wherein each of the plurality of tension members is disposed at least in part in a corresponding conduit, the conduit disposed on or within the item of apparel.

8. The item of apparel of claim 7, wherein the item of apparel is made of a material having a coefficient of friction and wherein each conduit has a lower coefficient of friction with respect to the tension members than the coefficient of friction of the material of the item of apparel with respect to the tension members.

9. The item of apparel of claim 7, wherein each conduit comprises at least in part a tube of material different from the matrix material of the item of apparel.

10. The item of apparel of claim 7, wherein the tension evener comprises a whippletree.

11. The item of apparel of claim 10, wherein the whippletree is a multi-tier whippletree.

12. The item of apparel of claim 10, wherein the whippletree comprises threadable members disposed for engaging with the substantially inextensible strands of the tension member.

13. The item of apparel of claim 12, wherein the threadable members include one or more of beads, blocks, rings, eyelets and whipple bars disposed for engaging with the plurality of substantially inextensible tension members, and wherein the one or more of beads, blocks, rings, eyelets and whipple bars is made of one of wood, metal, and a polymer, wherein polymer threadable members are one of injection-molded and three-dimensionally printed.

14. The item of apparel of claim 10, wherein the whippletree comprises at least one loop of one of substantially inextensible fabric and substantially inextensible cable disposed for engaging with the plurality of tension members.

15. The item of apparel of claim 1, wherein the item of apparel is a fit-adjustable garment.

16. The item of apparel of claim 1, wherein the item of apparel comprises a fit-adjustable harness.

17. The item of apparel of claim 1, wherein the item of apparel is a fit-adjustable orthopaedic brace.

18. A garment configured to be disposed upon a body of a wearer, the garment comprising:
At least one location-specific apparatus physically attached to the garment in a desired location corresponding to a target location on the body of the wearer of the garment;
a tension evener engaged with the location-specific device and comprising substantially inextensible tension members; and
a tensioner arranged to adjust a tension in the tension evener, the tension evener disposed and arranged to even tension in the tension members and maintain the location-specific apparatus at the target location.

19. The garment of claim 18, wherein the location-specific device is an orthopaedic brace.

20. The garment of claim 18, wherein the location-specific device is at least one of a heating and a cooling device.

21. The garment of claim 18, wherein the location-specific device is an inflatable bladder.

22. The garment of claim 18, wherein the location-specific device is a biometric device.

23. The garment of claim 18, wherein the location-specific device is a sensor.

24. The garment of claim 18, wherein the location-specific device is a harness.

25. The garment of claim 18, wherein the location-specific device is a safety device.
